(19) United States
(12) Patent Application Publication
(10) Pub. No.: US 2016/0235368 A1
(43) Pub. Date: Aug. 18, 2016

(54) DEVICE, METHOD AND SYSTEM FOR PROCESSING A PHYSIOLOGICAL SIGNAL

(71) Applicant: KONINKLIJKE PHILIPS N.V., Eindhoven (NL)

(72) Inventor: ANTONIUS HERMANUS MARIA AKKERMANS, EINDHOVEN (NL)

(21) Appl. No.: 15/025,742

(22) PCT Filed: Sep. 22, 2014
(86) PCT No.: PCT/EP2014/070068

§ 371 Date: Mar. 29, 2016

(30) Foreign Application Priority Data
Oct. 1, 2013 (EP) 13186865.5

(51) Int. Cl. A61B 5/00; A61B 5/0456; A61B 5/046; A61B 5/0245; A61B 5/024; A61B 5/08

(52) U.S. Cl. CPC A61B 5/7203; A61B 5/7221; A61B 5/02416; A61B 5/0816; A61B 5/046; A61B 5/0245; A61B 5/0456

(57) ABSTRACT

A device (10) for processing a physiological signal (11) is presented. The device comprises a feature detector (20) for detecting occurrences of a waveform feature of the physiological signal (11), wherein the physiological signal (11) is descriptive of a physiological process, and for providing a feature signal (23) descriptive of the detected occurrences of the waveform feature of the physiological signal (11), a debouncer (30) for removing non-indicative occurrences from the feature signal (23) that occur within a predetermined time window with respect to another occurrence and for providing a debounced feature signal (33), and an interpolator (40) for determining a baseline signal (12) by deriving values indicative of the physiological signal (11) at desired occurrences of the waveform feature, wherein desired occurrences are indicated by the debounced feature signal, and for interpolating the baseline signal (12) in-between said desired occurrences. Furthermore, a corresponding system, a method, a computer-readable non-transitory storage medium and a computer program are presented.

### FIELD OF THE INVENTION

The present invention relates to the field of determining vital signs of a subject, and in particular to a device, method and system for processing a physiological signal.

### BACKGROUND OF THE INVENTION

Vital signs of a subject are powerful indicators in determining a medical condition or fitness of a subject. Vital signs include, but are not limited to, respiratory rate (RR) and heart rate (HR) or pulse rate. The vital signs are descriptive of an underlying physiological process such as heartbeats or a respiratory movement. A physiological signal descriptive of the underlying physiological process can be measured and evaluated.

Reliable and accurate estimation of instantaneous frequencies of physiological processes, such as heart rate or respiratory rate, is critical for many health care applications. However, a robust estimation is especially challenging when novel unobtrusive sensors, such as plethysmographic (PPG) sensors or movement sensors, are used for continuous health monitoring in uncontrolled environments, e.g. a daily life setting. In these environments, sensors can generate significant amounts of potentially unreliable data. Therefore, in general, the signal processing gives problems during these disturbances that result in erroneous outputs. Nowadays many wrist-based biosensors for measuring a physiological signal are also equipped with accelerometers to estimate the movements and correct the measured signal based thereon.

State-of-the-art methods for respiration rate and heart rate detection are based on frequency-domain analysis or on continuous wavelength transforms (CWT). These methods are performed on segments of the physiological signal which comprise several occurrences of the underlying physiological phenomenon, for example multiple respiration cycles or a plurality of heartbeats. These segments are also referred to as windows and a corresponding signal processing is also referred to as windowed signal processing. A problem involved with this type of signal processing is latency. Due to the length of the processing windows, for example a thirty second window, the results are only available with a delay that corresponds to the length of the processed window. However, this latency is not acceptable in emergency situations. Furthermore, if a movement artifact is detected, the entire window is discarded.

U.S. 2013/0080489 A1 discloses a device and method for determining physiological information from a plurality of autocorrelation sequences. A continuous wavelet transformation can be applied to the autocorrelation sequence for determining respiration information. The autocorrelation is calculated for a processing window of e.g. 45 seconds duration. The plurality of autocorrelation sequences are generated based on a plurality of morphology metric signals. A morphology metric signal in turn is generated based on a photoplethysmograph (PPG) signal.

U.S. 2011/0301477 A1 discloses a device for providing biofeedback information to a subject. The device comprises a receiver for receiving heart rate data from a sensor. Types of sensors include a microphone (audio heart signals), pressure sensor (pulse pressure), electrocardiogram (ECG), photoplethysmography (PPG), as well as non-contact sensors that utilize RF or camera technologies. Regarding artifacts, the document teaches that windowed data is demeaned, which removes the DC offset of the data, and de-trended, which removes any underlying trend of the data. In terms of latency, the document teaches that shorter data windows yield faster updates of a subject's physiological state. In practical applications, latencies down to five seconds can be found.

However, there is a need to further reduce the latency. Moreover there is a need to check the integrity or quality of the signal with almost no latency and to determine whether a vital sign extraction is reliable or not. In particular in acute emergency situations, such as reanimation, performing cardiopulmonary resuscitation (CPR) or using an automated external defibrillator (AED), a low latency is crucial. Moreover, an efficient implementation, low memory usage and low power consumption are desirable.

### SUMMARY OF THE INVENTION

It is an object of the present invention to provide a device, method and system for processing physiological signals with reduced latency. It is a further object to provide an efficient implementation of a device for processing a physiological signal, advantageously having a low memory usage and power consumption.

In a first aspect of the present invention a device for processing a physiological signal is presented that comprises
a feature detector for detecting occurrences of a waveform feature of a received physiological signal, wherein the physiological signal is descriptive of a physiological process, and for providing a feature signal descriptive of the detected occurrences of the waveform feature of the physiological signal,
a debouncer for removing non-indicative occurrences from the feature signal that occur within a predetermined time window with respect to another occurrence and for providing a debounced feature signal, and
an interpolator for determining a baseline signal by deriving values indicative of the physiological signal at desired occurrences of the waveform feature, wherein desired occurrences are indicated by the debounced feature signal, and for interpolating the baseline signal in-between said desired occurrences.

In a further aspect of the present invention a system for processing a physiological signal is presented that comprises a concatenation of a first and a second device for processing the physiological signal as described above, wherein an input of the feature detector of the second device is connected to an output of the first device.

In yet further aspects of the present invention, there are provided a corresponding method, a computer program which comprises program code means for causing a computer to perform the steps of the method disclosed herein when said computer program is carried out on a computer as well as an non-transitory computer-readable recording medium that stores therein a computer program product, which, when executed by a processor causes the method disclosed herein to be performed.

Preferred embodiments of the invention are defined in the dependent claims. It shall be understood that the claimed method, system, computer program and medium have similar and/or identical preferred embodiments as the claimed device and as defined in the dependent claims.

The inventors have found that de-meaning or de-trending, as suggested in U.S. 2011/0301477 A1, fails to correct for movement artifacts that occur when, for example, using a PPG sensor. A PPG signal can be heavily distorted by movement artifacts since a slight displacement of the optical sensor may change the output signal considerably. This artifact is often rather a short spike or jump in the measured signal, optionally followed by a completely different signal amplitude, which cannot be corrected by de-trending the signal. In such situations the entire processing window may be classified as being bad and will be lost in state-of-the-art devices using processing windows.

It should be noted that also the use of an accelerometer for correction of artifacts has significant limitations since very small movements of an optical PPG sensor with respect to the skin can give huge signal deviations that are not proportional to a measured acceleration.

The present invention is further based on the finding that state-of-the-art methods are too slow in emergency situations. Even though U.S. 2011/03014477 A1 suggests that it can be run in real-time on mobile devices, the latency of this method is still limited to the length of the processing windows. The minimum duration for de-trending or de-meaning the data is defined by the processing window.

One element of the device according to the present invention is therefore to employ a feature detector for detecting occurrences of a waveform feature of the physiological signal. Thereby, characteristics of the waveform are identified as they occur. Waveform features include, but are not limited to peaks, dips, values exceeding a predetermined threshold, local maxima or minima, peaks/dips of a predetermined energy, slope or the like. The occurrences of the waveform feature can be provided as a feature signal at an output of the feature detector for further processing. Thus, the feature signal is provided with very low latency on a feature-by-feature basis. The feature signal thus indicates points in time or sample numbers, where a waveform feature is detected.

Experiments have shown that not all detected occurrences of a waveform feature can actually be attributed to an underlying physiological phenomenon but may be due to artifacts, e.g. a displacement of a PPG sensor. The device for processing a physiological signal according to an aspect of the present invention therefore comprises a debouncer for removing non-indicative occurrences from the feature signal. A non-indicative occurrence occurs within a predetermined time window with respect to another occurrence. For example, when measuring the heart rate, an occurrence of a peak immediately following a previous peak within a time window that is shorter than the minimum time for subsequent heartbeats of humans can be discarded. Such occurrences could be indicative of an artifact and are thus not desired. A debounced feature signal descriptive of the potentially desired occurrences can be provided at an output of the debouncer. The debounced feature signal provides desired occurrences since non-indicative occurrences have been removed.

The device according to the present invention further comprises an interpolator for determining a baseline signal by deriving values indicative of the physiological signal at desired occurrences of the waveform feature and interpolating the baseline signal in-between said desired occurrences. Thereby, the baseline signal can be determined on a feature-by-feature basis. Hence, the latency reduces to the interval between two features. This is a considerable advantage over state-of-the-art window-based solutions, since there is no need to wait for an entire window but only for the next feature.

A baseline signal can thus be defined by values of a physiological signal at occurrences of features. For example, the baseline signal represents an upper envelope if peaks are connected, or a lower envelope if dips are connected. Optionally a baseline can be determined by averaging the baselines obtained from one or more different types of features. Since the availability of the baseline signal depends on the occurrences of the waveform feature, the baseline is refreshed more quickly for a fast physiological process such as a beating heart and at a lower speed such as respiration.

A further advantage of the present invention is that the footprint of the corresponding implementation is very small, i.e. it is very efficient in that not much memory is used and few computations such as multiplications are required.

In an embodiment, the feature detector is configured to detect occurrences of at least one of peaks, dips, values exceeding a predetermined threshold, local maxima or minima, peaks/dips of a predetermined energy, slope of the physiological signal. For example peaks can be detected as values exceeding a predetermined threshold. Alternatively, a derivative of the physiological signal is evaluated for determining local minima or maxima. Optionally, a plurality of features, for example peaks and dips are detected such that a plurality of baselines can be detected to further increase the accuracy and reliability of the measurement. Optionally, peaks and dips are detected for carrying out balanced signal processing based on peaks and dips.

In a further embodiment, the debouncer and/or the interpolator is configured to provide the output signal in real time. Real time in this context means that the output is provided as soon as the next valid occurrence of a waveform feature is available. Thus, the output signal of the debouncer, i.e., the debounced feature signal, and/or the output signal of the interpolator, i.e. the baseline signal, can be provided on a feature-by-feature basis. The advantage of this real-time processing is that output signals can be provided with low latency which is especially beneficial in emergency situations.

In an embodiment, the debouncer is further configured to determine a time interval between two occurrences of features in the feature signal or in the debounced feature signal. A signal descriptive of time intervals between two occurrences of features in the feature signal or in the debounced feature signal can optionally be provided as a time interval signal at an output of the debouncer. These output signals are again available with low latency on a feature-by-feature basis. For the case of processing a heartbeat signal, wherein a feature corresponding to a heartbeat is detected, the time interval can also be referred to as an inter-beat-interval (IBI). Optionally, the time intervals of multiple features can be evaluated and optionally averaging applied to further reduce an error. However, averaging increases the latency.

In a further refinement of this embodiment, the debouncer is an adaptive debouncer wherein the length of the time window is adaptable depending on the time interval between two features. An advantage of this embodiment is that the time window of the debouncer can be adapted to a desired physiological phenomenon, for example for such as cardiac activity or respiration. The time interval between two subsequent features, for example the inter-beat-interval, does not change instantaneously due to the physiology of the subject. For example, upon performing a strenuous activity, the heart rate will gradually increase but not instantaneously. This improves the suppression of movement artifacts. For example at low heart rates, a longer time window can be used and more artifacts may fall within the adjusted time window.

In a further refinement of this embodiment, the length of the time window is shorter than half of the time interval between two successive features. Advantageously, a time interval between 0.1 to 0.5, preferably between 0.2 and 0.5, preferably between 0.3 and 0.5, preferably between 0.4 . . . 0.5 of the time interval between two successive features is selected as a length of the time window. Choosing a length longer than half of the time interval carries the risk that every second correct waveform feature is filtered out and that only half of the correct waveform features are detected. As an exemplary result only half of the actual heart rate or respiration rate might be detected. It should be noted that the given range is substantially different from windowed signal processing, wherein an interval time can be compared with an average time interval between two successive features. It should be noted that either the feature signal or the debounced feature signal can be evaluated. Selecting the time window as defined herein as shorter than half of the time interval between two successive features is advantageous since it enables the use of a debounced signal.

In a further embodiment, the feature detector is a peak detector comprising a delay element for comparing the physiological signal with a delayed signal derived from the physiological signal. An advantage of this embodiment is that the detection of features is independent of a signal magnitude. For an efficient implementation, a single storage site for storing a previous value of the physiological signal is sufficient.

In a further embodiment, the feature detector is a peak detector comprising a filter and a switch. Advantageously, a first order filter is used for a computationally efficient implementation.

In an embodiment, the device for processing a physiological signal further comprises a baseline removal unit configured to calculate a difference between the physiological signal and the baseline signal and to provide a baseline-removed physiological signal. It should be noted that the baseline signal can be calculated on a feature-by-feature basis and is thus available with low latency. Hence, a baseline removed signal for further processing can be provided with low latency. A further advantage of this embodiment is that a signal shape or morphology of an underlying physiological signal can be preserved when correcting disturbances. The prior art document U.S. 2011/03014477 A1, for example, suggests the use of a filter stage for correction. However, such filtering not only cancels undesired contributions but may also affect the signal shape. Furthermore, such a filter fails to suppress artifacts having a frequency component that lies within a frequency range expected for the physiological signal. The baseline-removed physiological signal is corrected for disturbances in the baseline regardless of their particular frequency.

In yet another embodiment, the device for processing a physiological signal further comprises a classification unit configured to determine a quality metric based on the baseline signal and/or the feature signal. For example, an amplitude of the baseline signal is evaluated. Advantageously, however, a derivative of the baseline signal or the feature signal is evaluated since this derivative is available on a feature-by-feature basis with low latency.

In an alternative embodiment, the device for processing a physiological signal comprises a classification unit configured to determine a quality metric based on a difference between two time intervals. For example a difference between two inter-beat-intervals (IBIs) is evaluated. As explained above, for example a heart rate does not change abruptly but increases or decreases continuously. Thus, the delta between two inter-beat-intervals should remain constant or have a slowly varying slope. This analysis is not possible in windowed signal processing. Optionally, however at the price of an increased latency, an average value of delta values can be computed. In this case, the quality metric would be available with a certain delay. It should be noted that even if a quality metric suffers from a certain delay the desired heart rate derived from the interval between two successive occurrences of a feature could still be provided with very low latency.

In a further embodiment, the device further comprises an evaluation unit configured to determine a vital sign signal based on the baseline signal and/or a time interval between two occurrences of features in the feature signal or in the debounced feature signal. For example, if a feature indicative of a heartbeat is detected, an instantaneous heart rate or instantaneous frequency of the heartbeats can be determined based on the time interval between two successive occurrences of features. In order to reduce erroneous measurement the debounced feature signal can be used.

### BRIEF DESCRIPTION OF THE DRAWINGS

These and other aspects of the invention will be apparent from and elucidated with reference to the embodiment(s) described hereinafter. In the following drawings:

FIG. 1 shows a schematic block diagram of an embodiment of a device for processing a physiological signal,

FIG. 2 shows a graph of exemplary signals,

FIG. 3 shows a schematic block diagram of a feature detector,

FIG. 4 shows a schematic block diagram of a sub-block of the feature detector,

FIG. 5 shows a schematic block diagram of an embodiment of a debouncer,

FIG. 6 shows a graph of intermediate signals and output signals of the feature detector,

FIG. 7 shows a graph of intermediate signals of the feature detector and an output signal of the debouncer,

FIG. 8 shows a schematic block diagram of a further embodiment of the device for processing a physiological signal,

FIG. 9 shows a graph of exemplary heart rate signals affected by a respiratory movement,

FIG. 10 shows a schematic block diagram of a concatenation of devices for processing a physiological signal,

FIG. 11 shows exemplary output signals of devices shown in FIG. 10,

FIG. 12 shows a flowchart of an algorithm for determining a respiration rate,

FIG. 13 shows an exemplary graph of output signals, and

FIG. 14 shows a further exemplary graph of output signals.

### DETAILED DESCRIPTION OF THE INVENTION

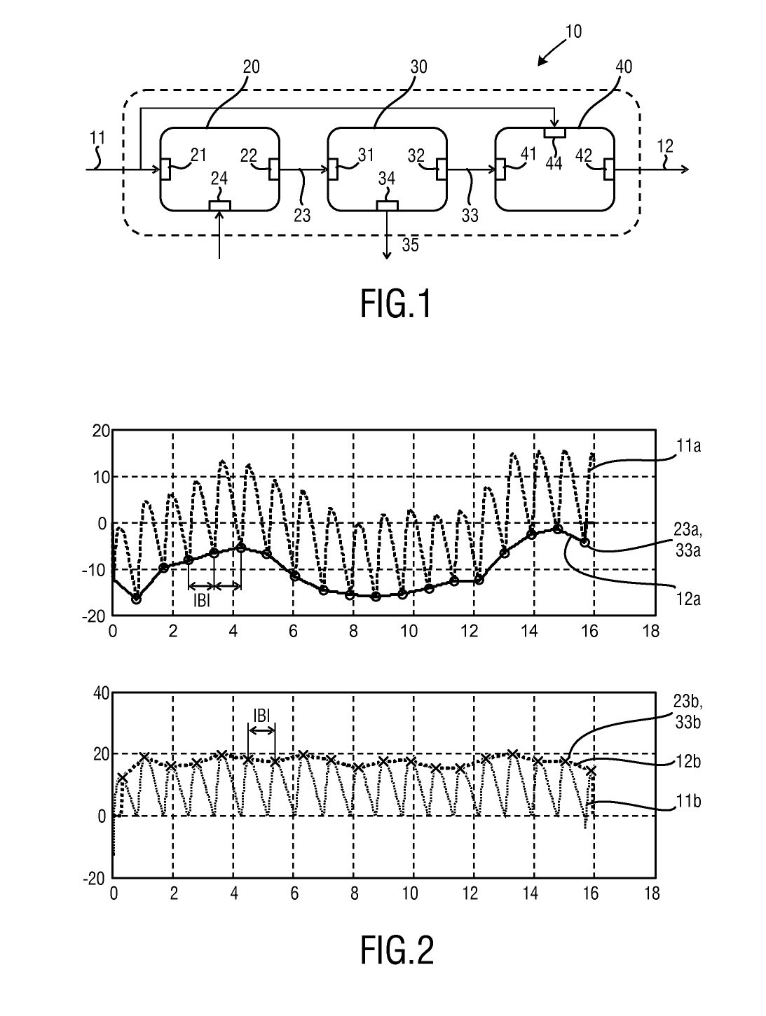

FIG. 1 shows a schematic block diagram of an embodiment of a device 10 for processing a physiological signal 11. The device 10 comprises a feature detector 20, a debouncer 30 and an interpolator 40 which provides a baseline signal 12.

The feature detector 20 is configured for detecting occurrences of a waveform feature of the physiological signal 11, wherein the physiological signal 11 is descriptive of a physiological process. Non-limiting examples of the physiological process are cardiac activity and respiration. Furthermore, the physiological signal 11 can comprise undesired disturbances, for example motion artifacts. Such a physiological signal can be obtained with different measurement techniques. In a preferred embodiment, a photoplethysmographic (PPG) sensor is used and the physiological signal 11 is a PPG signal. Optionally, preprocessing is applied to obtain the physiological signal 11 such as changing the sample rate, pre-filtering or combining multiple signals, for example fusing measurement signals from different axes of an accelerometer into a physiological signal.

The feature detector 20 comprises a feature detector input 21 for receiving the physiological signal 11 and a feature detector output 22 for providing a feature signal 23 descriptive of the detected occurrences of a waveform feature of the physiological signal 11.

In this exemplary embodiment, the feature detector is configured to detect occurrences of peaks and/or dips of the physiological signal 11. Optionally, the feature detector 20 comprises a selector input 24 for selecting the waveform feature that shall be detected. For example, the signal received at the selector input 24 of the feature detector 20 indicates whether a peak or dip of the physiological signal 11 shall be detected.

Alternatively, the feature detector 20 is configured to detect a different waveform feature such as a value exceeding a predetermined threshold, a local minimum or maximum, a slope, a predetermined curve shape, a peak of a given energy and the like. An occurrence of a waveform feature in this examples denotes a time or sample index, at which the waveform feature has been detected in the physiological signal. A feature signal 23 descriptive of the detected occurrences of the waveform feature of the physiological signal 11 is provided at an output 22 of the feature detector 20. It should be noted that the content of the feature signal 23 is not limited to the occurrences but can further comprise for example the values of the physiological signal 11 at the occurrences of the waveform feature.

The debouncer 30 is configured to remove non-indicative occurrences from the feature signal 23 that occur within a predetermined time window with respect to another occurrence. The debouncer 30 comprises a debouncer input 31 for receiving the feature signal 23 and a debouncer output 32 for providing a debounced feature signal 33. Optionally, the debouncer 30 is further configured to determine a time interval between two occurrences of features in the feature signal 31 or in the debounced feature signal 33. A signal descriptive of the time intervals can be provided as a time interval signal 35 at a time interval output 34 of the debouncer 30. Optionally, the time intervals of multiple features are evaluated and provided separately or combined, for example by averaging, at the time interval output 34. Further details of an exemplary embodiment of a debouncer 30 will be explained further below with reference to FIG. 5.

The interpolator 40 is configured to determine a baseline signal 12 by deriving values indicative of the physiological signal at desired occurrences of the waveform feature and interpolating the baseline signal in-between said desired occurrences. The interpolator 40 has an interpolator input 41 for receiving the debounced feature signal 33 from the debouncer 30 and an interpolator output 42 for providing the baseline signal 12. Optionally, the interpolator 40 further comprises an input 44 for receiving the physiological signal 11. Thereby, the interpolator 40 is provided with values of the physiological signal for deriving values indicative of the physiological signal 11 at the desired occurrences of the waveform feature. Alternatively, values of the physiological signal 11 or values indicative of the physiological signal at desired occurrences of the waveform feature can also be provided in the feature signal 23 and debounced features signal 33 which is received at the interpolator input 41.

FIG. 2 shows two exemplary graphs of a physiological signal 11 as the input of the device 10 for processing a physiological signal 11 and a baseline signal 12 as the output of the device 10.

Referring to FIG. 2 upper graph, the physiological signal 11a is descriptive of a beating heart. The horizontal axis denotes a time in seconds, whereas the vertical axis denotes an amplitude of the received physiological signal 11a. In this embodiment, the feature signal 23a is descriptive of detected occurrences of dips or local minima of the physiological signal 11a. The feature signal 23a is indicated by circles. Since the feature signal 23a in this embodiment does not comprise any non-indicative occurrences, the debounced feature signal 33a corresponds to the feature signal 23a. The baseline signal 12a is obtained by a linear interpolation in-between the values of the physiological signal 11a at the desired occurrences of the dips, thus the occurrences of the waveform feature dip, indicated by the debounced feature signal 33a.

Referring to FIG. 2 lower graph, the physiological signal 11b also represents a beating heart. In this embodiment, the feature detector is configured to detect occurrences of peaks of the physiological signal 11b. The occurrences of the peaks are provided as a feature signal 23b which is denoted by crosses in FIG. 2 lower graph. Also in this embodiment, the debounced feature signal 33b corresponds to the feature signal 23b since no non-indicative occurrences, that occur within a predetermined time window with respect to another occurrence, have been determined by the debouncer. The baseline signal 12b in this embodiment is determined by taking the values of the physiological signal 12b at occurrences of the peaks and interpolating the values in-between said peaks.

The baseline signal 12a in FIG. 2, upper graph, can be seen as a lower envelope of the physiological signal 11a, whereas the baseline signal 12b in FIG. 2, lower graph, can be seen as an upper envelope of the physiological signal 11b. It should be noted that different types of interpolation can be used such as a spline interpolation or cubic interpolation.

The time interval between two successive occurrences of features in the feature signal 23a, 23b or in the debounced feature signal 33a, 33b in this embodiment is denoted as the inter-beat-interval (IBI) which indicates a temporal separation between successive dips (FIG. 2, upper graph) or peaks (FIG. 2, lower graph). The inverse of the IBI indicates an instantaneous frequency of heartbeats, i.e., an instantaneous heart rate. In general, such a time interval between two occurrences of a waveform feature can indicate an instantaneous frequency of a physiological phenomenon. An advantage of the device 10 for processing a physiological signal 11 according to an aspect of the present invention is that the instantaneous frequency is available as soon as the feature detector 20 detects a following occurrence of the waveform feature. Thus, there is not need for waiting until a time window or data segment has been acquired that can be processed in frequency domain.

Not only the instantaneous frequency, but also the baseline signal can be provided on a feature-by-feature basis with low latency. Advantageously, the proposed device 10 allows a cost effective and efficient implementation, since there is no need to store lengthy signal traces but only very short recordings.

Furthermore it should be noted that the physiological signal 11b of FIG. 2, lower graph, can be obtained as the difference between the physiological signal 11a and the baseline signal 12a of FIG. 2, upper graph. Thus, to generate a clean or baseline-removed physiological signal, the baseline signal can be subtracted from the original physiological signal. Obviously, the original physiological signal 11a (FIG. 2, upper graph) is band-limited according to the Nyquist criterion and may contain frequencies up to half the sample frequency. In practice, the analog Nyquist filter could be set to, for example, 10 Hz to generate a proper physiological signal 11a. A first stage of the signal processing (FIG. 1, upper graph) can give a first measurement of the IBI time interval and therefore a first heart rate. In an optional second stage, the same scheme of FIG. 1 can be applied to the baseline removed physiological signal 11b (FIG. 2, lower graph). The output of the interpolator then gives the envelope or contour signal of the peaks 12b of the baseline removed physiological signal 11b which can be defined as the envelope of the original physiological signal 11a. In this second stage, the IBI frequency can optionally again be determined to have a second measurement of the periodicity time of the original physiological signal.

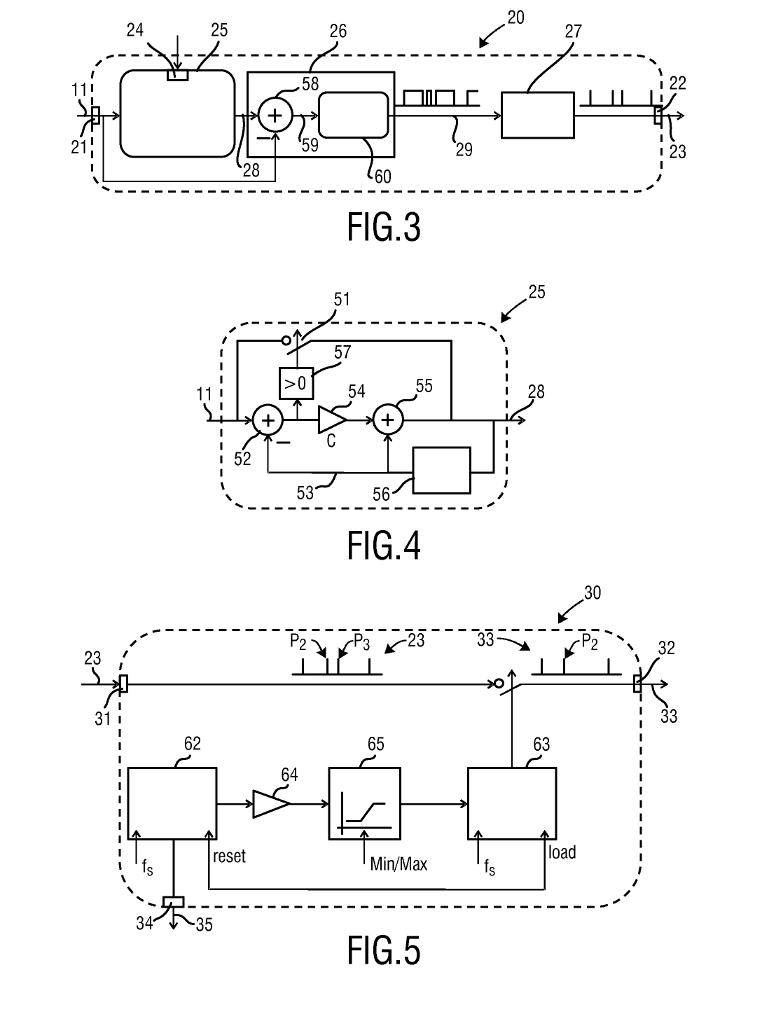

FIGS. 3 and 4 provide further details about an exemplary embodiment of the feature detector 20. FIG. 5 provides further details about an exemplary embodiment of the debouncer 30.

FIG. 3 shows an exemplary embodiment of a feature detector 20. The feature detector 20 comprises a conditional filter 25, a comparator 26 and a transition detector 27. The physiological signal 11 is received at an input of the conditional filter 25 and the feature signal 23 provided at an output of the transition detector 27.

A more detailed block diagram of an embodiment of the conditional filter 25 is shown in FIG. 4. The conditional filter 25 is formed by first order filter and a switch 51. The filter characteristic is determined by the coefficient C and can be set to appropriate values for example for heart rate detection, respiration rate detection, or another desired quantity. For heart rate detection, practical values are set for a frequency between fc=0.5 Hz and for respiration detection fc=0.2 Hz. The filter coefficient C is determined by C=2pi*fc/fs, where fs is the sampling rate and fc is the desired cut-off frequency.

In FIG. 4, the physiological signal 11 is provided at an input of an adder 52, where an intermediate signal form a feedback loop 53 is subtracted and the result passed on with a factor determined by stage 54 to a second adder 55. The second adder 55 adds the output of the intermediate stage 54 to the signal from the feedback loop 53 to provide an output signal 28 of the conditional filter 25. The feedback path comprises a delay stage 56 that feeds back the output signal 28 to the first adder 52 and the second adder 55. The switch 51 in the upper part of FIG. 4 is controlled by a comparator 57. The comparator 57 can close the switch 51 to directly forward the physiological signal 11 from the input of the conditional filter to the output 28. The output of the first adder 52 serves as the input for the comparator 57. For detecting a peak, the comparator 57 is configured to determine whether the output of the adder 52 is >0, for detecting a dip the comparator 57 is configured to determine whether the output of the adder 52 is <0 and then closes the switch 51. A control signal for configuring the conditional filter 25 for peak or dip detection can be provided via the selector input 24 shown in FIG. 3. An advantage of this peak or dip detector system is that the detection characteristics become independent of a gain or magnitude of the physiological signal 11. This is important, since a signal magnitude can vary considerably between different subjects, for different positioning of the sensor or after (unintentional) movement of the sensor.

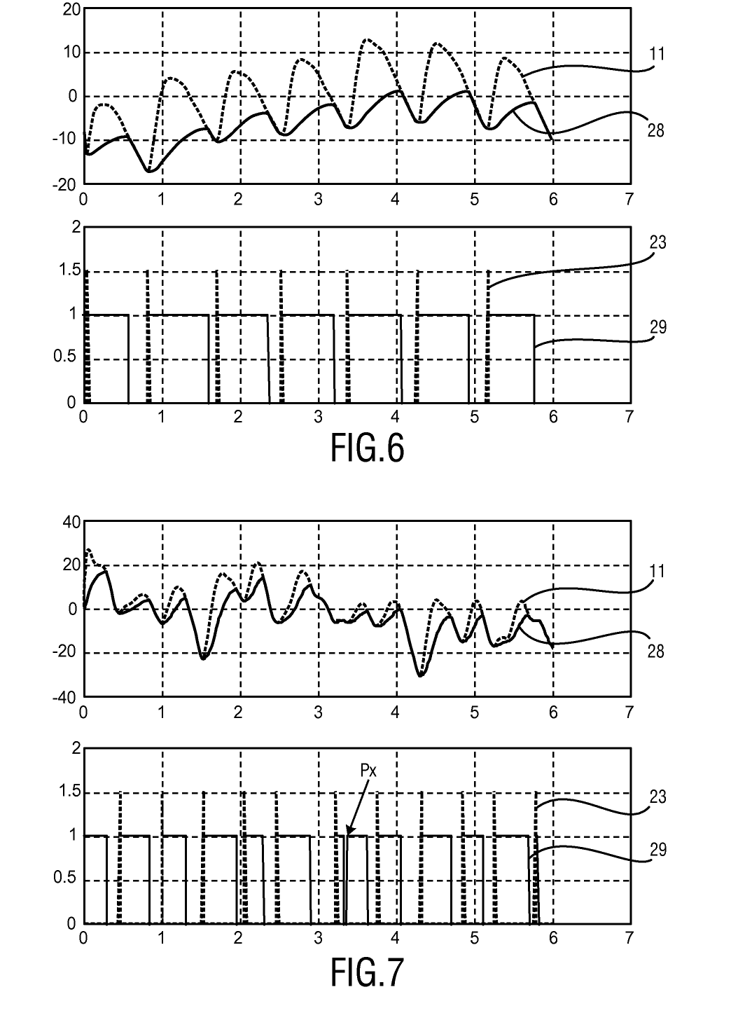

FIG. 6 and FIG. 7 upper graph show typical output signals 28 of the conditional filter 25 upon receiving a physiological signal 11 at the input. In this exemplary embodiment, the conditional filter 25 is configured for detecting dips, i.e. the comparator 57 is set for closing the switch 51 if the output of the adder 52 is <0. As can be seen from FIG. 6, FIG. 7 upper graph, for the first part of each wave of the physiological signal 11, the result of the filter elements (52, 53, 54, 55, 56 in FIG. 4) is provided as the output signal 28, whereas during a second part of each waveform, the switch 51 is closed and the physiological signal 11 as the input signal of the conditional filter 25 is directly provided as the output signal 28.

Referring again to FIG. 3, the output 28 of the conditional filter 25 is provided as a first input to the comparator 26. The physiological signal 11 is provided as a second input. In a first step, the physiological signal 11 is subtracted from the output 28 of the conditional filter 25 by adder (subtractor) 58. The output 59 of the adder 58 is provided as an input to a sign detector 60 for determining a sign of its input signal 59. This results in the block-shaped signal 29 in FIGS. 6 and 7, lower graph. If the physiological signal 11 is larger than the output 28 of the conditional filter 25 (in this case configured for detecting dips) a logic high is provided as the output signal 29 of the comparator 26. The output 29 of the comparator 26 is thus a digital signal as shown in FIGS. 6, 7 lower graph. In a next step, the transition detector 27 detects up-going transition, FIG. 6, lower graph, which are then provided as the output 23 of the feature detector 20 and indicate occurrences of detected dips.

As an alternative to the comparator 26 as a separate block, the output of the comparator 57 of the conditional filter 25, shown in FIG. 4, or its logic inverse could directly be used as an input to the transition detector.

FIG. 5 shows an exemplary embodiment of a debouncer 30. The debouncer 30 receives the feature signal 23 at a debouncer input 31 and provides a debounced feature signal 33 at a debouncer output 32. In this embodiment, the up-going transitions of the feature signal are debounced and “cleaned” if transitions occur within a predetermined time window with respect to another occurrence. Such occurrences can be due to for example noise on the physiological signal or movement artifacts. As shown in the insets above the signal path from the input 31 to the output 32, the peak P3 that follows peak P2 in the feature signal 23 is removed such that peak P3 is not present anymore in the debounced feature signal 33. FIG. 7 lower graph, curve 33, shows this clean-up or discarding of the extra spurious transition at about 3.3 seconds indicated by Px. In other words, the debounced feature signal 33 in FIG. 7 lower graph only indicates occurrences of a dip in the physiological signal 11, where no further dip follows within a predetermined time window.

Referring again to the exemplary embodiment of the debouncer 30 in FIG. 5, the debouncer 30 comprises a switch 61. If the switch is closed, the input signal is forwarded to the output, otherwise, the input is discarded. The debouncer further comprises a period time counter 62 implemented as an up-counter and a debounce counter 63 implemented as a down-counter. The period time counter 62 is reset via a reset input by a logic high at the debounced feature signal 33. Alternatively, the reset input of the period time counter 62 can be configured to be reset by the feature signal directly. The period time counter 62 further comprises a clock input for receiving clock signals at a sampling frequency fs of the physiological signal. Thus, the clocked period time counter 62 increments the counter and determines the time between two signals at a reset input and provides the result at an output 34 as the time interval signal 35. The counter value of the period time counter 62 is further provided to a debounce window element 64 for determining a length of a debounce window. Optionally, a limiter 65 receives the output of the debounce window element 64 to determine whether the debounce window is within given minimum and maximum range and provides the limited debounce window as a load input to the debounce counter 63. Thus, the load input can be a value with which the debounce counter 63 can be initialized upon receiving a logic high signal from the debounced feature signal at its “load” input. The debounce counter also comprises an input for a clock signal, which in this embodiment is the sampling frequency fs. Upon receiving clock signals at the fs input, the debounce counter counts down and closes the switch 61 upon reaching zero. This way, non-indicative occurrences in the feature signal 23 that occur within a predetermined time window, i.e. predetermined by the load value of the debounce counter and the clock frequency fs, with respect to another occurrence are discarded and thus removed from the feature signal to provide the debounced feature signal 33.

Thus, the debouncer 30 in this embodiment is an adaptive debouncer, wherein the debounce window depends upon the period time between two transitions, i.e. occurrences of a feature in the feature signal or the debounced feature signal. In other words, the adaptive debouncer 30 determines the period time between two successive peaks or dips and further generates the time stamps for the occurrences of these maxima or minima. Based on the previous period time measured by the period time counter 62 the current debounce time window will be set to discriminate incoming new transition pulses. A fraction of the previous period time is used to set the debounce window. In practice, this value is about 0.5 for heart rate pulses detection and for respiration this is set to 0.3. The larger the debounce window, for example closer to 0.5 of the period time, the debouncer becomes more discriminative or selective in filtering out bad or undesired transitions.

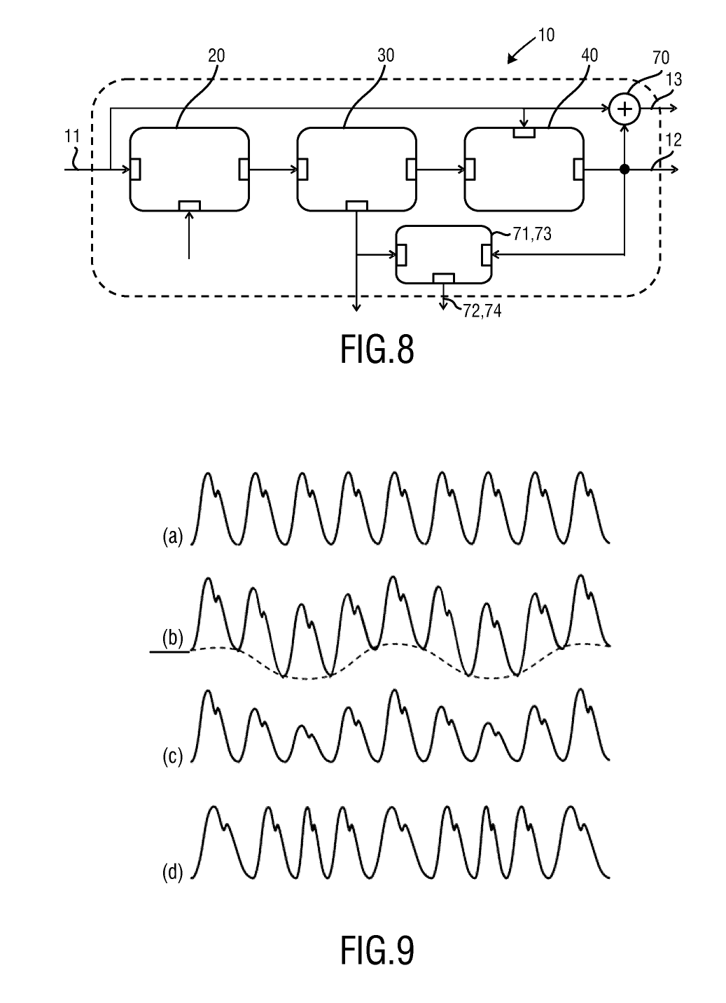

FIG. 7 lower graph shows a debounced feature signal 33, wherein a transition of the signal 29 occurring at 3.3 seconds, indicated by Px, has been discarded. FIG. 8 shows an alternative embodiment of a device 10 for processing a physiological signal 11. In addition to the elements shown in and described with reference to FIG. 1, the device 10 according to this embodiment further comprises an optional baseline removal unit 70, a classification unit 71 and/or evaluation unit 73. The classification unit and the evaluation unit can optionally be implemented as one block as shown in FIG. 8. The baseline removal unit 70 is configured to calculate the difference between the physiological signal 11 and a baseline signal 12 and to provide a baseline removed physiological signal 13. It should be noted that the baseline removed physiological signal 13 can again serve as an physiological signal for a following device for processing a physiological signal. This is illustrated by curve 11b in FIG. 2, lower graph, wherein curve 11b is the difference between the physiological signal 11a and the baseline signal 12a in FIG. 2, upper graph.

In an embodiment, the classification unit 71 is configured to determine a quality metric 72 based on a magnitude of the baseline signal 12. Alternatively, the classification unit 71 is configured to receive the feature signal 23 or the debounced feature signal 33 and to determine the quality metric 72 based thereon. Further alternatively, the classification unit is configured to determine a quality matric 72 based on a difference between two time intervals from the time interval signal 35. The respective signals can be obtained from the debouncer 30. For example, a difference or delta between two inter-beat-intervals (IBI) can be evaluated. This delta should remain rather constant, since a continuously increasing or decreasing heart rate has a slowly varying slope. For example, the heart rate typically does not jump from 60 beats per minute (bpm) to 120 bpm within a second but increases towards this value. For a healthy subject a threshold of allowable delta values of 20 bpm is practical. However, a patient-specific threshold could be set. Delta values can be also be evaluated upon recovery of the subject. Thereby, also a rate of recovery can be identified. Further, a large delta value can be indicative of a movement artifact or measurement error.

In an embodiment, the evaluation unit 73 is configured to determine a vital sign signal 74 based on the baseline signal 12 and/or a time interval between two occurrences of features in the feature signal 23 or in the debounced feature signal. For example, a heart rate can be determined from the time interval signal 35. An advantage of this evaluation is a very low latency, since the inter-beat-intervals are available on a feature-by-feature basis. Thus, instead of an average heart rate, an instantaneous heart rate can be provided on a beat-by-beat basis. Furthermore, the baseline signal 12 or baseline-removed physiological signal 13 can be evaluated. It should be noted that the evaluation unit can optionally be configured to combine feature-by-feature analysis with conventional windowed signal processing. For example, the instantaneous heart rate is determined on a feature-by-feature basis from the time interval between to occurrences of features in the feature signal 23, whereas the respiration rate is determined by frequency-domain analysis from the baseline signal 12.

FIG. 9 shows four exemplary graphs of a photoplethysmographic (PPG) signal obtained as the physiological signal 11. It is known from literature (e.g. Addison et al.,: “Developing an algorithm for pulse oximetry derived respiratory rate (RRoxi): a healthy volunteer study”, Journal of Clinical Monitoring and Computing (2012)) that the respiration can modulate a PPG signal in three different ways as illustrated in FIGS. 9(b) to (d).

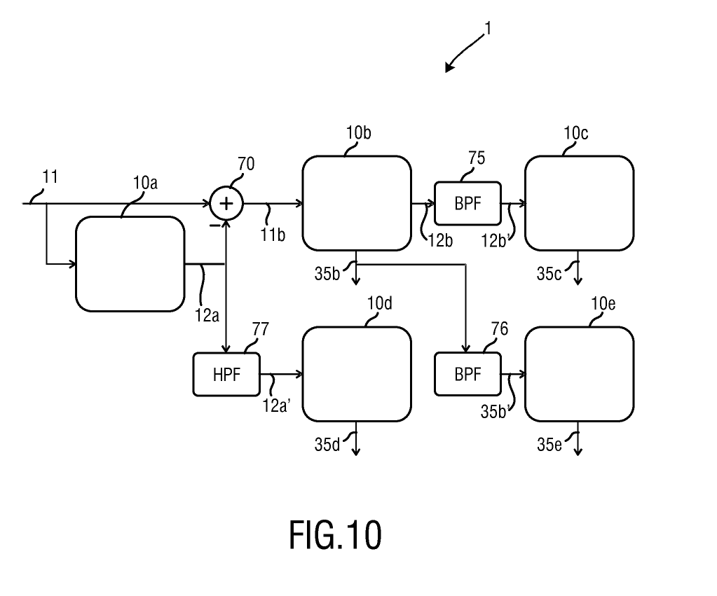

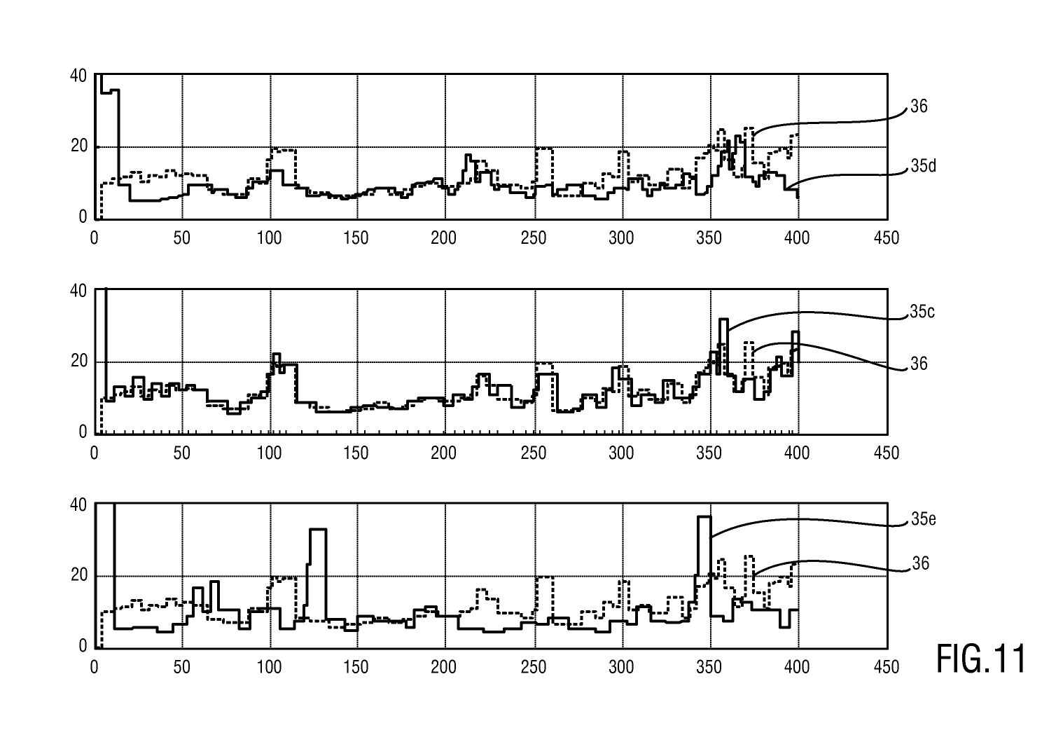

FIG. 9(a) shows an unmodulated cardiac pulse waveform as the physiological signal 11. FIG. 9(b) shows a baseline modulation wherein cardiac pulses are riding on top of a baseline. The baseline is shown as a dashed line. FIG. 9(c) illustrates an amplitude modulation of the PPG signal, wherein cardiac pulse amplitudes vary over respiration cycle. FIG. 9(d) illustrates a respiratory sinus arrhythmia (RSA), wherein the inter-beat-interval (IBI), varies over respiration cycles. It is possible to determine the respiration rate by evaluating one or more of these phenomena as will be exemplarily shown with reference to FIG. 10. Of course, the heart rate can also be determined.

FIG. 10 shows an advantageous concatenation of a plurality of devices for processing a physiological signal wherein each of the devices 10a to 10e in principle correspond to the device 10 for processing a physiological signal according to FIG. 1 or 8. The system 1 receives the physiological signal 11 as an input. A skilled person will select the relevant blocks if not all outputs are desired.

The exemplary embodiment shown in FIG. 10 shows a scheme for robust heart rate and pulse rate variability detection as well as a method to detect a respiration rate. Application examples are atrial fibrillation detection, respiration detection in hospital general wards or in pulse detection and assistance during cardiopulmonary resuscitation (CPR) events. The system comprises multiple basic devices as described with reference to FIG. 1 or 8. The signal processing is done in time domain and is robust for input scaling or magnitude of all inputs and has low latency times.

For improved robustness some optional additional filters, for example second order Butterworth filters, can be applied. The additional high-pass filters (HPF) and band-pass filters (BPF) blocks have high cut-off frequencies of 1 Hz. The low cut-off frequency is, for example, 0.05 Hz. These optional additional filters could be simplified or even be discarded. The frequency response will be tailored to the desired application, for example heart rate or respiration rate detection.

The system 1 receives the raw physiological signal 11 as an input. In this context, a device 10a-10e for processing a physiological signal is also referred to as a “baseline extractor”. In the first baseline extractor, the feature detector is configured for detecting dips of the signal. The baseline signal 12a provided at an output of the baseline extractor 10a corresponds to the baseline signal 12a of FIG. 2, upper graph. The system 1 further comprises a baseline removal unit 70, wherein the baseline signal 12a is subtracted from the raw physiological signal 11 for obtaining a baseline-removed physiological signal 11b, corresponding to 11b in FIG. 2, lower graph. This signal serves as an input for the second baseline extractor 10b. A first output of the baseline extractor 10b provides a time interval signal 35b that is descriptive of the inter-beat-intervals (IBI) of successive heartbeats and is therefore indicative of the instantaneous heart rate. A second output of the baseline extractor 10b provides a baseline signal 12b, corresponding to baseline signal 12b in FIG. 2 lower graph, that is provided via a first band-pass filter 75 to a third baseline extractor 10c. This third baseline extractor 10c evaluates the time interval between successive peaks and the time interval signal between successive dips of the filtered baseline signal 12b′, calculates an average between the time interval between peaks and dips and provides the result as a time interval signal 35c. This signal is descriptive of an envelope or amplitude based respiration, see FIG. 11 middle graph, as indicated by FIG. 9(c). The respiration rate determined with a respiration belt (respiband) 36 is shown in all graphs of FIG. 11 for reference. The horizontal axis denotes a time in seconds, whereas the vertical axis denotes a measured respiration rate in breaths per minute.

The time interval signal 35b of baseline extractor 10b is further provided to a further baseline extractor 10e via a second band-pass filter 76. The baseline extractor 10e receives the filtered time interval signal 35b′, and determines a time interval signal 35e therefrom. The time interval signal 35e is descriptive of the respiration based on evaluating a heart rate variability or frequency modulation, see FIG. 11 lower graph, as indicated by FIG. 9(d).

The output 12a of the first baseline extractor 10a is further provided to another baseline extractor 10d via a high pass filter 77. Based on the filtered baseline signal 12a′, the baseline extractor 10d determines a time interval signal 35c which provides a baseline based respiration, see FIG. 11 upper graph, as indicated by FIG. 9(b).

In conclusion, the same device architecture according to an aspect of the present invention (FIG. 1 or 8) can be used for evaluating all three different ways of how the respiration modulates the physiological PPG signal 11.

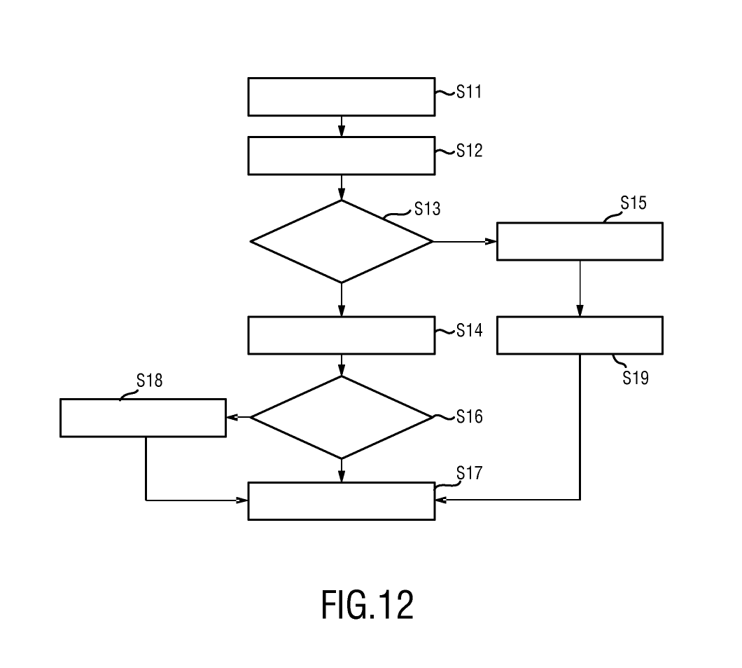

The quality of the extracted vital sign can be further improved by combining time-domain processing with frequency-domain processing as illustrated with reference to FIG. 12 for the example of estimating a respiration rate. Other flow diagrams are possible that also realize this concept. Advantageously, time-domain information of an envelope of the physiological signal is combined with frequency-domain information of a frequency modulation, for example due to a respiratory sinus arrhythmia (RSA). The inventors have found that in some situations for some modulation types the time-domain breath-to-breath respiration rate may underestimate the actual respiration rate. Thus, to further improve the results, frequency-domain analysis can be used in addition to time-domain analysis to calculate the respiration rate. In a good situation, these values correspond with time-domain analysis. From experiments and measurements on humans, it is concluded that mostly envelope detection (FIG. 11, middle graph) works best for real-time breath-to-breath respiration rate estimation with root mean square (RMS) inter-beat-interval (IBI) errors smaller than 2% at rest and one minute average time. On a breath-to-breath level, thus with low latency, RMS errors of less than 1.5 breaths have been found.

To avoid underestimation of the respiration rate and to come to a more robust respiration rate extraction, FIG. 12 shows a flow chart that gives an improved estimate of the respiration rate. However, in this case the latency is again related to the window size for frequency-domain analysis, for example about 30 seconds in practical applications. For continuous monitoring, in most applications a small latency is allowable. However, in life-threatening situations latency times should be as small as possible. In particular for pulse detection during reanimation (CPR) a low latency time is crucial. The system disclosed herein combines quickly providing measurements on a feature-by-feature basis with the slower, however in some applications, more accurate frequency-domain analysis.

The system of FIG. 10, now comprising the baseline extractors 10a to 10c, is again used to extract the respiration rate on a breath-to-breath basis. The device 10c provides an interval signal 35c that is descriptive of the respiration period time. In case of respiration rate extraction the time constants of the peak detector with its conditional filter and/or the debounce window of adaptive debouncer are adjusted to a proper value for respiration.

Referring again to FIG. 12, in step S11 the respiration rate is calculated in real-time using a baseline extractor 10c as described with reference to FIG. 10. In a second step S12, a spectral density or frequency spectrum of the signal 12b (see also FIG. 2), is calculated for a time frame of for example 30 seconds. In a third step S13, a decision is made whether the signal quality is good or bad. The process continues with step S14 for good signal quality and with step S15 for bad signal quality. In step S14, the highest peak frequency, i.e. the peak with the highest frequency, is extracted.

In a next decision step S16, the frequency extracted in step S14 is compared with the respiration rate determined in step S11. If the extracted frequency and the heart rate correspond, the method proceeds to step S17 and ends. If the extracted frequency and the heart rate do not match, the frequency determined in step S14 is set as the respiration rate in step S18 and the procedure ends in step S17.

If a poor signal quality has been determined in step S13, the method proceeds with step S15, where the heart rate spectral density for a time frame of for example 30 sec. is determined. Based on this spectrum, the highest peak frequency is extracted. The spectrum represents the heart rate variability. The extracted highest peak frequency is set as the respiration rate in step S19. The process again ends in step S17.

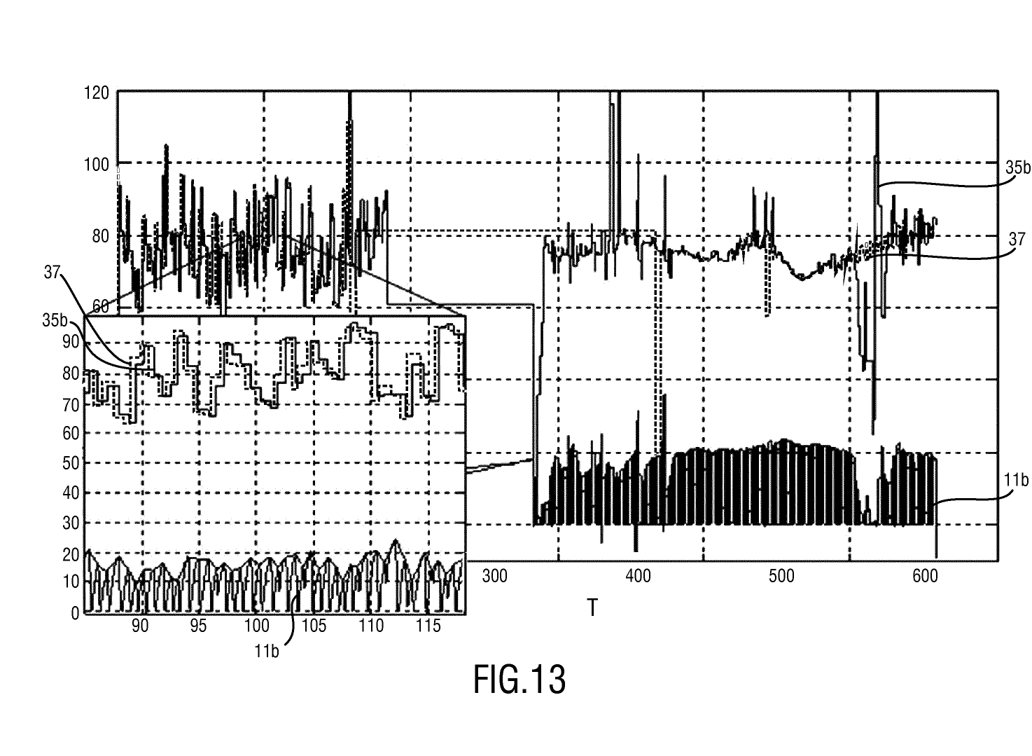

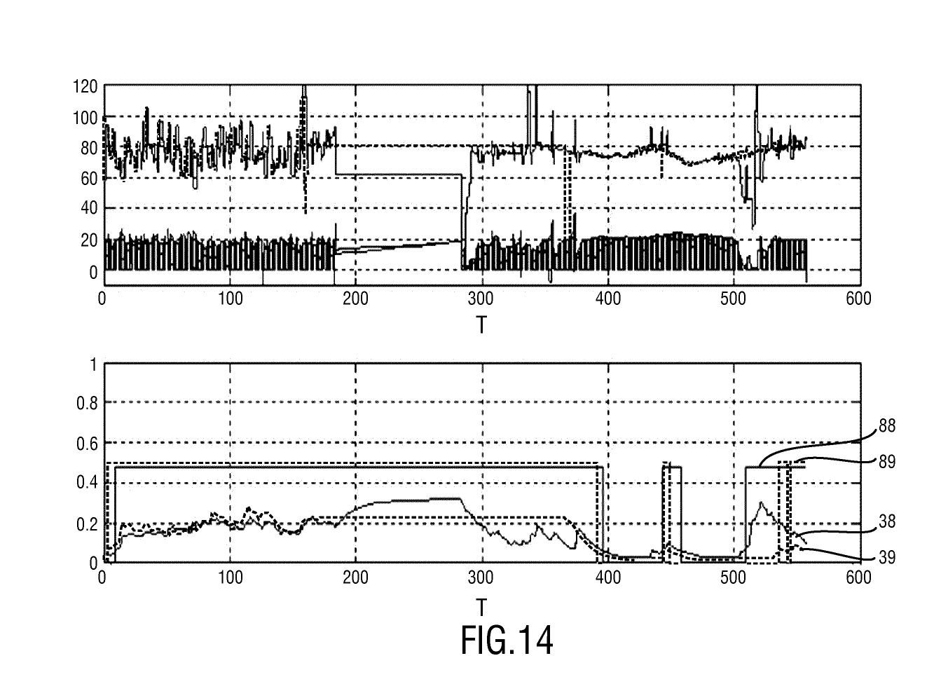

Referring to FIGS. 13 and 14, the inventors have found that the inter-beat-interval (IBI) time measurements or pulse rate variability based on the device and system according to the present invention is very accurate compared with heart rate variability (HRV) measurements based on ECG and can be used for detection of heart arrhythmias like atrial fibrillation (AF). Detection of paroxysmal or “silent” atrial fibrillation is one of the most important risk indicators for cardio health risk or strokes. Therefore, unobtrusive sensing of AF in daily life situations is desirable. The method and algorithms according to this invention have been tested on atrial fibrillation patients which gives errors smaller than 5% RMS value at one minute averaging during AF events compared with the ECG as a reference. During non-AF events the error is generally smaller.

FIG. 13 shows an ECG 37/PPG 35b recording before and after cardio version, so before and after the AF event. The horizontal axis denotes a time in seconds, whereas the vertical axis denotes an instantaneous heart rate. The figure clearly shows the resemblance of the heart rate measured with ECG 37 and the heart rate measured with PPG 35b according to an aspect of the present invention during AF. The lower curve in FIG. 13 indicates the baseline removed physiological signal 11b from FIG. 10, whereas the upper curve indicates the time interval signal 35b from FIG. 10 that is indicative of the instantaneous heart rate. For reference the ECG-based heart rate signal 37 is depicted.

FIG. 14 shows the detection of AF based on the heart rate variability 38 determined with the system according to the present invention compared with a heart rate variability 39 determined based on the R-R interval of the ECG. FIG. 14 upper graph repeats the graph of FIG. 13 without inset. FIG. 14 lower graph shows a first curve of the heart rate variability 38 based on the heart rate 35b and a second curve 39 of the heart rate variability based on the R-R interval of the ECG 39 shown as curve 37 in FIG. 13. The respective variabilities in curves 38, 39 are compared with a threshold to illustrate the detection of an AF event. Curve 88 denotes PPG-based detection of AF, whereas curve 89 denotes AF detection based on ECG.

In conclusion, a device, method and system for processing a physiological signal have been presented, wherein the physiological signal can be evaluated with low latency. Thereby, a reliable and accurate estimation of instantaneous frequencies of physiological rhythms such as heart rate or respiratory rate becomes possible which is critical for many health care applications, in particular in emergency situations. Furthermore, the use of sensors that can be applied in an unobtrusive way in everyday life situations becomes feasible. In these environments, sensors can create significant amount of potentially unreliable data. Therefore, a robust flexible estimation of feature-to-feature intervals for these signals is proposed. The method does not require any prior knowledge about the morphology of the analyzed waveforms and can thus be easily applied to a variety of different physiological signals and measurement modalities. The invention is not limited to extract in real-time a beat-to-beat heart rate and a breath-to-breath respiration rate from a photoplethysmographic physiological signal as exemplarily shown herein but can be applied to many more signals where such physiological information is contained. Moreover, in addition to information about vital signs, also quality metrics and baseline correction to conceal signal errors due to movement artifacts are provided.

A skilled person is aware that aspects of the present invention can be implemented as hardware elements, a combination of hardware and software or in software, for example executed on a multi-purpose microcontroller or other processing device.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

A computer program may be stored/distributed on a suitable non-transitory medium, such as an optical storage medium or a solid-state medium supplied together with or as part of other hardware, but may also be distributed in other forms, such as via the Internet or other wired or wireless telecommunication systems.

Any reference signs in the claims should not be construed as limiting the scope.

1. A device (10) for processing a physiological signal (11) comprising:
a feature detector (20) for detecting occurrences of a waveform feature of a received physiological signal (11), wherein the physiological signal (11) is descriptive of a physiological process, and for providing a feature signal (23) descriptive of the detected occurrences of the waveform feature of the physiological signal (11),
a debouncer (30) for removing non-indicative occurrences from the feature signal (23) that occur within a predetermined time window with respect to another occurrence and for providing a debounced feature signal (33), and
an interpolator (40) for determining a baseline signal (12) by deriving values indicative of the physiological signal (11) at desired occurrences of the waveform feature, wherein desired occurrences are indicated by the debounced feature signal, and for interpolating the baseline signal (12) in-between said desired occurrences.

2. The device according to claim 1,
wherein the feature detector (20) is configured to detect occurrences of at least one of peaks, dips, values exceeding a predetermined threshold, local maxima or minima, peaks/dips of a predetermined energy, slope of the physiological signal.

3. The device according to claim 1,
wherein said debouncer (30) and/or said interpolator (40) is configured to provide the output signal (33, 12) in real-time.

4. The device according to claim 1,
wherein said debouncer (40) is further configured to determine a time interval (IBI) between two occurrences of features in the feature signal (23) or in the debounced feature signal (33).

5. The device according to claim 4,
wherein said debouncer is an adaptive debouncer (40) wherein the length of the time window is adaptable depending on the time interval (IBI) between two features.

6. The processing apparatus according to claim 5,
wherein the length of the time window is shorter than half of the time interval (IBI) between two successive features.

7. The device according to claim 1,
wherein said feature detector (20) is a peak detector comprising a delay element (56) for comparing the physiological signal (11) with a delayed signal (53) derived from the physiological signal (11).

8. The device according to claim 1,
wherein said feature detector (20) is a peak detector comprising a filter and a switch (51).

9. The device according to claim 1,
further comprising a baseline removal unit (70) configured to calculate a difference between the physiological signal (11a) and the baseline signal (12a) and to provide a baseline-removed physiological signal (11b).

10. The device according to claim 1,
further comprising a classification unit (71) configured to determine a quality metric based on the baseline signal (12) and/or the feature signal (23).

11. The device according to claim 4,
further comprising a classification unit (71) configured to determine a quality metric based on a difference between two time intervals (IBI).

12. The device according to claim 1,
further comprising an evaluation unit (73) configured to determine a vital sign signal (74) based on the baseline signal (12) and/or a time interval (IBI) between two occurrences of features in the feature signal (23) or in the debounced feature signal (33).

13. A system (1) for processing a physiological signal comprising a concatenation of a first and a second device (10a-10e) according to claim 1, wherein an input of the feature detector of the second device (10c, 10e) is connected to an output of the first device (10b).

14. A method for processing a physiological signal comprising the steps of:
detecting occurrences of a waveform feature of a received physiological signal (11), wherein the physiological signal (11) is descriptive of a physiological process, and providing a feature signal (23) descriptive of the detected occurrences of the waveform feature of the physiological signal (11),
removing non-indicative occurrences from the feature signal (23) that occur within a predetermined time window with respect to another occurrence and providing a debounced feature signal (33), and
determining a baseline signal (12) by deriving values indicative of the physiological signal (11) at desired occurrences of the waveform feature, wherein desired occurrences are indicated by the debounced feature signal, and interpolating the baseline signal (12) in-between said desired occurrences.

15. Computer program comprising program code means for causing a computer to carry out the steps of the method as claimed in claim 14 when said computer program is carried out on the computer.
